(19) United States
(12) Patent Application Publication
(10) Pub. No.: US 2024/0296930 A1
(43) Pub. Date: Sep. 5, 2024

(54) INTERACTIVE, COMPUTER-BASED SYSTEM OF APPLYING ACUPRESSURE

(71) Applicant: AURORA STARCHILD, SACRAMENTO, CA (US)

(72) Inventor: AURORA STARCHILD, SACRAMENTO, CA (US)

(21) Appl. No.: 18/117,053

(22) Filed: Mar. 3, 2023

(51) Int. Cl. G16H 20/30; G09B 19/00

(52) U.S. Cl. CPC G16H 20/30; G09B 19/003

(57) ABSTRACT

The invention provides an interactive, computer-based method of applying acupressure. More specifically, step by step instructions are provided to user for the application of acupressure to a patient or to oneself (i.e., self-acupressure).

### FIELD OF THE INVENTION

The invention provides an interactive, computer-based method of applying acupressure. More specifically, step by step instructions are provided to user for the application of acupressure to a patient or to oneself (i.e., self-acupressure).

### BACKGROUND OF THE INVENTION

The practice of acupressure is ancient. Its invention is believed to originate amongst the ancient Asian peoples in China, Japan and Southeast Asia where it continues to be practiced in earnest.

Acupressure is the application of pressure on specific points on the body to control symptoms such as pain or digestive issues. It is used in some kinds of massage therapy like “Shiatsu” massages. It can also be done as part of an acupuncture treatment, or you can do acupressure on yourself.

Acupressure is an alternative medicine technique often used in conjunction with acupuncture or reflexology. In treatment, physical pressure is applied to acupressure points which may be applied by hand, by elbow, or with various devices.

Some current medical studies suggest that acupressure is effective at helping to manage nausea, vomiting, insomnia, low back pain, migraines, and constipation, among other things.

Until most recently, Western medicine mostly eschewed and scoffed at the helpfulness and efficacy of Eastern healing techniques like acupressure, acupuncture and others. Now, Western hospitals and mainstream medical insurance plans often allow for their use and inclusion within treatment plans. Western medical schools (and other educational institutions) have even begun to teach their use and provide certifications for practitioners who learn and master their techniques.

Given such use and advancing popularity, practitioners, whether lay or professional, require teaching tools applicable to a modern world. Use of computers, computer networks, the Internet, wireless connections and more enable teaching of a range of fields that make learning more available to more people. The marriage of learning viable Eastern healing techniques like acupressure to distribute through computer-based technology is an inventive solution whose time has come.

The several embodiments enclosed herein provide novel solutions for the teaching and application of acupressure for practitioners and self-application for patients.

### SUMMARY OF THE INVENTION

Accordingly, the invention provides an interactive, computer-based system of applying acupressure to a partner which comprises a computerized device that has a central processing unit, memory and operational programming. More specifically, it comprises at least one central processing unit, non-transitory memory in operational connection with the at least one central processing unit, and one or more algorithms (i.e., computer programs) which are written onto the non-transitory memory having instructions for operation of the one or more central processing units.

The invention herein further comprises an interactive, computer-based system of applying acupressure to a partner comprising a computerized device that has all of the following:
i. At least one central processing unit;
ii. Non-transitory memory in operational connection with the central processing unit;
iii. One or more algorithms (computer programs) written onto the non-transitory memory and having instructions for operation of the central processing unit; and
iv. Acupressure learning software.
   The acupressure learning software provides all of the following steps:
i. It provides a tutorial about the basics of acupressure to a user;
ii. It provides a tutorial about the location of acupressure points to a user;
iii. It provides a tutorial for the correct touch of acupressure to a user;
iv. It identifies a present health issue in the partner through prompts provided by the computerized device;
v. It identifies a part of the partner's body presenting the health issue through prompts provided by the computerized device;
vi. It identifies one or more acupressure points in the partner's body corresponding to the part of the partner's body presenting the health issue through prompts provided by the computerized device;
vii. It identifies an appropriate point of pressure along the identified one or more acupressure points corresponding to the partner's body presenting the health issue through prompts provided by the computer-based system; and
viii. It applies a requisite amount of pressure to the one or more acupressure points for a requisite amount of time to affect relief to the partner's health issue through prompts provided by the computer-based system.

Also provided herein is an interactive, computer-based method of applying self-acupressure, comprising a computerized device having a central processing unit, memory, operational programming, and software that provides an acupressure learning tool to a user. The tool has the following steps:
It provides a tutorial about the basics of acupressure;
It provides a tutorial about the location of acupressure points;
It provides a tutorial for the correct touch of acupressure;
A user self-identifies a present health issue in the user through prompts provided by the computerized device;
A user self-identifies a part of the user's body presenting the health issue through prompts provided by the computerized device;
A user self-identifies one or more acupressure points in the user's body corresponding to the part of the user's body presenting the health issue through prompts provided by the computerized device;
A user self-identifies an appropriate point of pressure along the identified one or more acupressure points corresponding to the user's body presenting the health issue through prompts provided by the computerized device; and
A user self-applies a requisite amount of pressure to one or more acupressure points for a requisite amount of time to affect relief to the user's health issue through prompts provided by the computerized device.

Also comprised may be a tutorial about the location of each of fourteen meridians in a human body.

Health issues impacted by use of the interactive, computer-based system herein include mental health issues, emotional health issues, physical sickness health issues, and/or physical pain health issues.

### BRIEF DESCRIPTION OF THE FIGURES

The various exemplary embodiments of the present invention, which will become more apparent as the description proceeds, are described in the following detailed description in conjunction with the accompanying drawings, in which:

FIG. 1 is a process flow sheet of the computer-based system for applying self-acupressure or acupressure to a partner.

### DETAILED DESCRIPTION OF THE INVENTION

The following description is provided as an enabling teaching of the present systems, and/or methods in its best, currently known aspect. To this end, those skilled in the relevant art will recognize and appreciate that many changes can be made to the various aspects of the present systems, and/or methods described herein, while still obtaining the beneficial results of the present disclosure. It will also be apparent that some of the desired benefits of the present disclosure can be obtained by selecting some of the features of the present disclosure without utilizing other features.

Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

As used throughout, the singular forms “a,” “an” and “the” include plural referents unless the context clearly dictates otherwise. Thus, for example, reference to “an element” can include two or more such elements unless the context indicates otherwise.

As used herein, the terms “optional” or “optionally” mean that the subsequently described event or circumstance can or cannot occur, and that the description includes instances where said event or circumstance occurs and instances where it does not.

The word “or” as used herein means any one member of a particular list and also includes any combination of members of that list. Further, one should note that conditional language, such as, among others, “can,” “could,” “might,” or “may,” unless specifically stated otherwise, or otherwise understood within the context as used, is generally intended to convey that certain aspects include, while other aspects do not include, certain features, elements and/or steps. Thus, such conditional language is not generally intended to imply that features, elements and/or steps are in any way required for one or more particular aspects or that one or more particular aspects necessarily include logic for deciding, with or without user input or prompting, whether these features, elements and/or steps are included or are to be performed in any particular aspect.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods.

Thus, if there are a variety of additional steps that can be performed it is understood that each of these additional steps can be performed with any specific aspect or combination of aspects of the disclosed methods.

The invention herein provides an interactive, computer-based method of applying self-acupressure which comprises a computerized device having a central processing unit, memory and operational programming. The computerized device is used to initiate an interactive process which includes all of the following steps:
a. Providing an acupressure learning tool to a user, the tool having the following steps;
   i. Providing a tutorial about the basics of acupressure;
   ii. Providing a tutorial about the location of acupressure points;
   iii. Providing a tutorial for the correct touch of acupressure;
b. Self-identifying a present health issue in said user through prompts provided by said computerized device;
c. Self-identifying a part of said user's body presenting said health issue through prompts provided by said computerized device;
d. Self-identifying one or more acupressure points in said user's body corresponding to said part of said user's body presenting said health issue through prompts provided by said computerized device;
e. Self-identifying an appropriate point of pressure along said identified one or more acupressure points corresponding to said user's body presenting said health issue through prompts provided by said computerized device; and
f. Self-applying a requisite amount of pressure to said one or more acupressure points for a requisite amount of time to affect relief to said user's health issue through prompts provided by said computerized device.

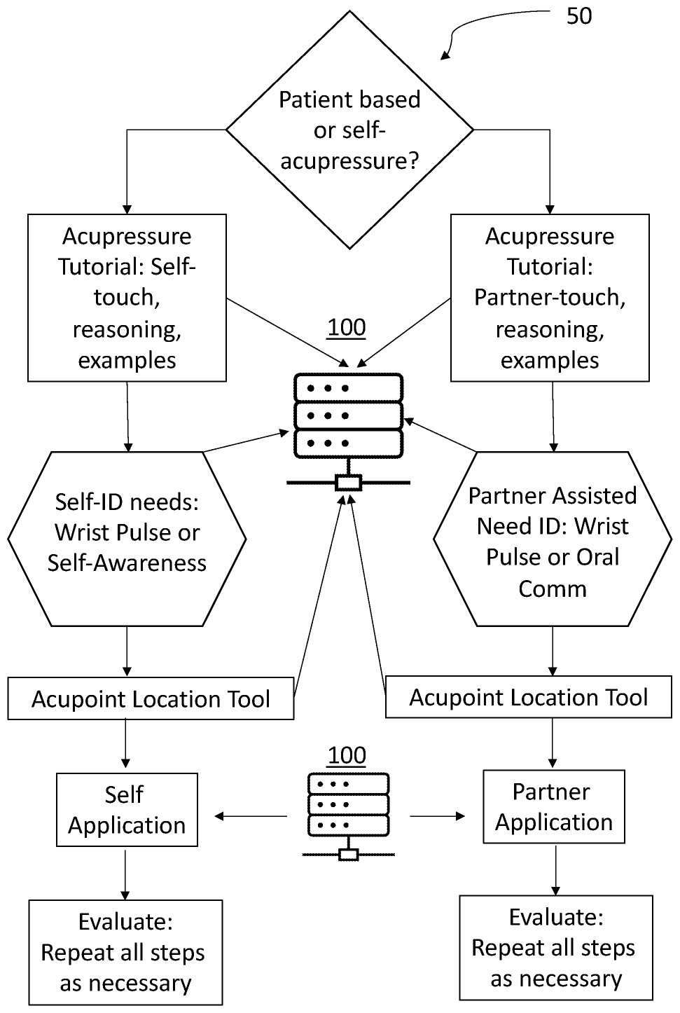

FIG. 1 provides a process flow sheet of the computer-based system for applying self-acupressure or acupressure to a partner. The process in FIG. 1 shows the process flow of computer-based system 50 (or “system 50”) of applying either self-acupressure or acupressure to a partner. The first determination for a patient is to determine who the patient might be, i.e., whether one self or another. This is the determination of whether a user of the system seeks to apply the system taught acupressure techniques to another or to one self. Once this determination is made, system 50 then guides the user through the remainder of the process.

Next, whether for self-application or application for another, system 50 provides specific instruction and/or tutorials to a user based upon user input. That is, user can interactively input into system 50 ailments, stresses, feelings and more to enable system 50 to direct a user to the appropriate part of the body to touch, the proper amount of pressure to apply and the right amount of time to engage acupressure. All of the foregoing are prescribed by system 50.

System 50 is powered by a computerized system 100 comprising one or more devices to operate interactively with a user. Devices included in computerized system 100 may be one or more personal mobile devices, pad devices, laptops, desktop computers and the like. Brands of such devices may derive from APPLE®, SAMSUNG®, TOSHIBA®, IBM®, IPAD® and the like. The foregoing list is not exhaustive, and persons of skill in the art will recognize that other like devices made by other manufacturers are within the scope of the use of system 50 herein.

Next provided is a wrist pulse analysis, either self or of a patient. This analysis is used to benchmark the health state of one's self or of a patient. The pulse is a physiological phenomenon that propagates throughout the arterial system and is regarded as a traveling pressure generated by the cardiac cycle. Conditions of poor health may cause variations in arterial properties, pulse related parameters including shape, rhythm, and strength and should be known prior to use of system 50 herein.

The acupoint location tool is provided by computer-based system 50, its function being to guide with specificity a user for a patient's care. The acupoint location tool is interactive and software based. It asks questions and guides use of acupressure until said acupressure is applied at the right place, in the right way for the right amount of time to affect the necessary result. This is true for either self or patient application thereof.

Once the software based acupoint location tool has been provided, acupressure is applied by a user to either one's self or to a patient; i.e., “self-application” or “partner application” as shown. After such application, user evaluation of success is required. If success has not been achieved, a user can inform system 50 thereof and recommence the steps as necessary with the iterative feedback and direction of system 50 once again.

Health issues impacted by use of the interactive, computer-based tutorial method herein include mental health issues, emotional health issues, physical sickness health issues, and/or physical pain health issues.

Whether the computer-based system and methods herein are used for acupressure applied to a patient or for self-acupressure, the benefits and advantages over a manual system therefor are several. First, a purely manual system (e.g., using a book with pictures) cannot ask questions. Second, a manual system cannot refine its answers based upon real time feedback from a user. Third, a manual system cannot provide real time adjustments nor adjust to new data not contemplated by a published work. Fourth, a manual system cannot, with highly specific accuracy, show in real time a part of the body for acupressure application or count with a user how long such acupressure should be provided.

It should be appreciated and understood that the present invention may be embodied as systems, methods, apparatus, computer readable media, non-transitory computer readable media and/or computer program products. The present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” The present invention may take the form of a computer program product embodied in one or more computer readable medium(s) having computer readable program code embodied thereon.

One or more computer readable medium(s) may be utilized, alone or in combination. The computer readable medium may be a computer readable storage medium or a computer readable signal medium. A suitable computer readable storage medium may be, for example, but not limited to, an electronic, magnetic, optical, electromagnetic, infrared, or semiconductor system, apparatus, or device, or any suitable combination of the foregoing. Other examples of suitable computer readable storage medium include, without limitation, the following: an electrical connection having one or more wires, a portable computer diskette, a hard disk, a random access memory (RAM), a read-only memory (ROM), an erasable programmable read only memory (EPROM or flash memory), an optical fiber, an optical storage device, a magnetic storage device, or any suitable combination of the foregoing. A suitable computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Python, C++ or the like and conventional procedural programming languages, such as the “C” programming language or similar programming languages. The program code may execute entirely on the user's computing device (such as, a computer), partly on the user's computing device, as a stand-alone software package, partly on the user's computing device and partly on a remote computing device or entirely on the remote computing device or server. In the latter scenario, the remote computing device may be connected to the user's computing device through any type of network, including a local area network (LAN) or a wide area network (WAN), or the connection may be made to an external computing device (for example, through the Internet using an Internet Service Provider).

The present invention is described herein with reference to flowchart illustrations and/or block diagrams, can be implemented by computer program instructions. These computer program instructions may be provided to a processor of a general purpose computing device (such as, a computer), special purpose computing device, or other programmable data processing apparatus to produce a machine, such that the instructions, which execute via the processor of the computing device or other programmable data processing apparatus, create means for implementing the functions/acts specified in the flowchart and/or block diagram block or blocks.

These computer program instructions may also be stored in a computer readable medium that can direct a computing device, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computing device, other programmable data processing apparatus, or other devices to cause a series of operational steps to be performed on the computing device, other programmable apparatus or other devices to produce a computer implemented process such that the instructions which execute on the computing device or other programmable apparatus provide processes for implementing the functions/acts specified in the flowchart and/or block diagram block or blocks.

It should be appreciated that the function blocks or modules shown in the drawings illustrate the architecture, functionality, and operation of possible implementations of systems, methods and computer program media and/or products according to various embodiments of the present invention. In this regard, each block in the drawings may represent a module, segment, or portion of code, which comprises one or more executable instructions for implementing the specified logical function(s). It should also be noted that, in some alternative implementations, the functions noted in the block may occur out of the order noted in the figures. For example, the function of two blocks shown in succession may, in fact, be executed substantially concurrently, or the blocks may sometimes be executed in the reverse order, depending upon the functionality involved.

It will also be noted that each block and combinations of blocks in any one of the drawings can be implemented by special purpose hardware-based systems that perform the specified functions or acts, or combinations of special purpose hardware and computer instructions. Also, although communication between function blocks or modules may be indicated in one direction on the drawings, such communication may also be in both directions.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. An interactive, computer-based system of applying acupressure to a partner comprising:
a. A computerized device having a central processing unit, memory and operational programming;
i. At least one central processing unit;
ii. Non-transitory memory in operational connection with said central processing unit;
iii. One or more algorithms (computer programs) written onto said non-transitory memory and having instructions for operation of said central processing unit;
b. Acupressure learning software comprising the following steps:
i. Providing a tutorial about the basics of acupressure to a user;
ii. Providing a tutorial about the location of acupressure points to a user;
iii. Providing a tutorial for the correct touch of acupressure to a user;
iv. Identifying a present health issue in said partner through prompts provided by said computerized device;
v. Identifying a part of said partner's body presenting said health issue through prompts provided by said computerized device;
vi. Identifying one or more acupressure points in said partner's body corresponding to said part of said partner's body presenting said health issue through prompts provided by said computerized device;
vii. Identifying an appropriate point of pressure along said identified one or more acupressure points corresponding to said partner's body presenting said health issue through prompts provided by said computer-based system; and
viii. Applying a requisite amount of pressure to said one or more acupressure points for a requisite amount of time to affect relief to said partner's health issue through prompts provided by said computer-based system.

2. The interactive, computer-based system of applying acupressure of claim 1 further comprising a tutorial about the location of each of fourteen meridians in a human body.

3. The interactive, computer-based system of applying acupressure of claim 1 wherein said health issues are at least partially mental health issues.

4. The interactive, computer-based system of applying acupressure of claim 1 wherein said health issues are at least partially emotional health issues.

5. The interactive, computer-based system of applying acupressure of claim 1 wherein said health issues are at least partially physical sickness health issues.

6. The interactive, computer-based system of applying acupressure of claim 1 wherein said health issues are at least partially physical pain health issues.

7. An interactive, computer-based method of applying self-acupressure, comprising:
a. A computerized device having a central processing unit, memory and operational programming;
b. Providing an acupressure learning tool to a user, the tool having the following steps;
i. Providing a tutorial about the basics of acupressure;
ii. Providing a tutorial about the location of acupressure points;
iii. Providing a tutorial for the correct touch of acupressure;
iv. Self-identifying a present health issue in said user through prompts provided by said computerized device;
v. Self-identifying a part of said user's body presenting said health issue through prompts provided by said computerized device;
vi. Self-identifying one or more acupressure points in said user's body corresponding to said part of said user's body presenting said health issue through prompts provided by said computerized device;
vii. Self-identifying an appropriate point of pressure along said identified one or more acupressure points corresponding to said user's body presenting said health issue through prompts provided by said computerized device; and
viii. Self-applying a requisite amount of pressure to said one or more acupressure points for a requisite amount of time to affect relief to said user's health issue through prompts provided by said computerized device.

8. The interactive, computer-based system of applying acupressure self-acupressure of claim 7 further comprising a tutorial about the location of each of fourteen meridians in a human body.

9. The interactive, computer-based system of applying acupressure self-acupressure of claim 7 wherein said health issues are at least partially mental health issues.

10. The interactive, computer-based system of applying acupressure self-acupressure of claim 7 wherein said health issues are at least partially emotional health issues.

11. The interactive computer-based system of applying acupressure self-acupressure of claim 7 wherein said health issues are at least partially physical sickness health issues.

12. The interactive, computer-based system of applying acupressure self-acupressure of claim 7 wherein said health issues are at least partially physical pain health issues.
